(19) United States
(12) Plant Patent Application Publication
(10) Pub. No.: US 2006/0041969 P1
(43) Pub. Date: Feb. 23, 2006

(54) ARGYRANTHEMUM FRUTESCENS PLANT NAMED 'ARGYDUPEA'

(50) ARGYRANTHEMUM FRUTSCENS; ARGYRANTHEMUM

(76) Inventor: Annemarie Houbraken, Enkhuizen (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(21) Appl. No.: 10/920,103

(22) Filed: Aug. 17, 2004

(51) Int. Cl. A01H 5/00

(52) U.S. Cl. PLT/263

(57) ABSTRACT

A distinct cultivar of Argyranthemum frustescens plant named ‘Argydupea’ characterized by its upright vigorous plant habit, good field performance, double pink flowers, dark green coloured leaves, freely flowering with numerous inflorescences per plant.

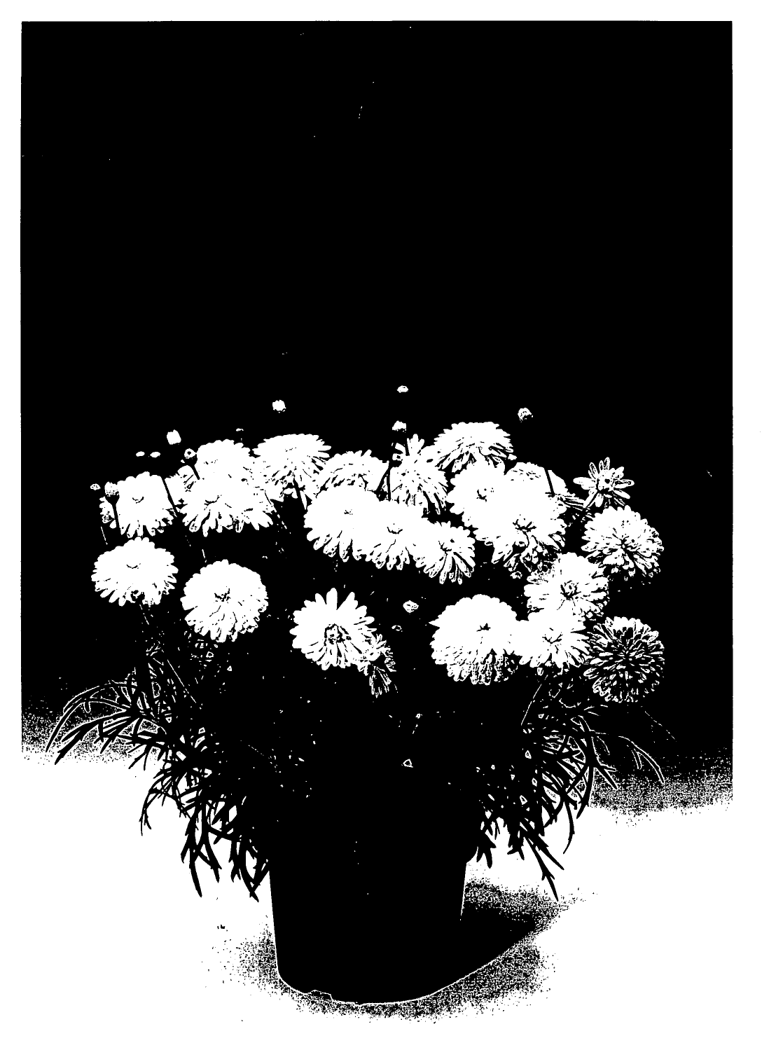

### LATIN NAME OF THE GENUS AND SPECIES OF THE PLANT CLAIMED Argyranthemum frutescens

### VARIETAL DENOMINATION

‘Argydupea’

### BACKGROUND OF THE NEW PLANT

The present invention relates to a new and distinct cultivar of Marguerite Daisy plant, botanically known as Argyranthemum frutescens and referred to by the cultivar name ‘Argydupea’. The new cultivar is a product of a planned breeding program conducted by the inventor in Enkhuizen, The Netherlands. The objective of the breeding program was to develop a more vigorous plant that flowers early with good field performance during the whole summer.

### DDESCRIPTION OF THE DRAWING

This new Marguerite Daisy plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Marguerite Daisy plant. The data which defines these characteristics were collected from asexual reproductions carried out in Enkhuizen, The Netherlands. The plant history was taken on 25 weeks old plants, grown in the field in Enkhuizen, The Netherlands with day temperatures ranging from 16 to 30 degree C., and night temperatures ranging from 10 to 16 degrees Celsius.

Color references are primarily to the RHS Color Chart of The Royal Horticultural Society of London where general terms of ordinary dictionary significance are used.

TABLE 1
Differences between the new cultivar ‘Argydupea’ and
three similar cultivars

| | | | | | ‘Summer | | | |
|---|---|---|---|---|---|---|---|---|
| | | | | | Melody’ | | | |
| | | | | | (U.S. Plant | | | |
| | | | ‘Sugar Lace’ | | Patent | | ‘Powder Puff’ | |
| | ‘Argydupea’ | | (Unpatented) | | No. 11,763) | | (Unpatented | |
| Plant Height | 30 | cm | 20 | cm | 25 | cm | 50 | cm |
| Plant Width | 60-70 | cm | 45 | cm | 45 | cm | 60 | cm |
| Foilage color | N138B | | 146A | | 146A | | 146A | |
| Flower color | 69B | | N155B | | 70A | | 75C | |
| Flower size | 2.3-3.5 | cm | 1.5 | cm | 2-2.5 | cm | 3-4 | cm |

| The Plant | | |
|---|---|---|
| Classification - Botanical | | Argyranthemum frutescens |
| | | cv. ‘Argydupea’ |
| Parentage | | |
| Female parent | | Proprietary seedling section |
| | | of Argyranthemum frutescens, |
| | | identified as number A 27-1. |
| | | Breeder's code of the new |
| | | plant is D 46-2 (unpatented). |
| Male parent | | Unidentified section of |
| | | Argyranthemum frutescens |
| | | (unpatented) |
| Propagation | | |
| Type cuttings | | Terminal cuttings |
| Time to intiated roots | | 7-10 days at air |
| | | temperature of 2l degrees C. |
| Time to develop roots | | 7-14 days at air |
| | | temperature of 18 degrees C. |
| Plant Description | | |
| Growth habit | | Upright |
| Plant height | | 25-28 cm |
| Vigor | | Vigorous |
| Speading area of plant | | 60-70 cm |
| Strength | | Very good |
| Branching character | | Freely branching, |
| | | plants do not require pinching |
| Crop time | | About 10 weeks are required |
| | | to produce a finished |
| | | flowering plant in a |
| | | 10.5 cm container from a good |
| | | developed cutting |
| The Stem | | |
| Diameter | | 3-5 mm |
| Shape | | Round, bit grooved |
| Anthocyanin pigmentation | | Absent |
| Length of internode | | 1-2 cm |
| Pubescence | | Absent |
| Color | | 145A |
| The Foliage | | |
| Arrangement | | Alternate |
| Shape of leaf | | Bipinnatisect |
| Leaf apex | | Acute |
| Leaf base | | Attenuate |
| Texture | | Thick glabrous smooth |
| Length | | 4.5-9 cm |
| Width | | 1.5-3.5 cm |
| Depth of incision | | Lanciniate |
| Color | Upper side | N138B |
| | Lower side | 147B |

| Pubescence | | Absent |
|---|---|---|
| Petiole | | Absent |
| Venation | Shape | Pinnate |
| | Color | N138B |

Flower Bud

| | | |
|---|---|---|
| Peduncle | Length | 5-10 cm |
| | Strength | Flexible |
| | Texture | Smooth |
| | Color | 144A |
| Size of the bud | Length | 3-5 mm |
| | Diameter | 4-5 mm |

| Shape | | Round |
|---|---|---|
| Color | | 164B |
| Inflorescence | | |
| Diameter | | 2.3-3.5 cm |
| Form | | Double flowered capitulum with |
| | | ± 100 ray florets with no |
| | | disc florets, which give the |
| | | double impression |
| Receptacle height | | 0.5 cm |
| Involucre/phyllaries | | 3 series of 5 bracts, |
| | | tightly to receptacle |
| Shape | | Elliptic, with broad apex |
| Margin | | Entire |
| Length | | 2-3 mm |
| Color | | Upper: 143A |
| | | Lower: 151A |
| Fragrance | | Absent |
| Quality of inflorescences | | ±100 flowers/plant depending |
| | | on age and growing |
| | | conditions |
| Flowering period | | April til October in |
| | | N-W Europe, plants flower |
| | | continuously during this period |
| Ray florets | | |
| Shape of ray florets | | Ligulate |
| Apex | | Rounded, little incised |
| Base | | Cordate |
| Margin | | Entire |
| Color | | 69B |
| No. of ray florets per inflorescene | | 110 |
| Size | Length | 0.9-1.1 cm |
| | Width | 0.4-0.5 cm |

| | |
|---|---|
| Texture | Smooth |
| Ray florets and androecium | Absent |
| Gynoecium | |
| Pistil number | One per floret, 110 per flower head |
| Pistil length | ±2 mm |
| Style color | Yellow green 144C |
| Style length | ±2 mm |
| Stigma color | Yellow orange 14A |
| Stigma shape | Bilobed |
| Ovary color | 144C |

### Seed

Seed production is not observed

### Disease Resistance

No susceptibility nor resistance to fungal, bacterial or viral pathogens has been noted. Material is free of virus and Chrysanthemum stunt viroid (CSVd).

What is claimed is:

1. A new and distinct variety of Argyranthemum frutescens plant, substantially as herein illustrated and described.
